(19) United States
(12) Patent Application Publication
(10) Pub. No.: US 2010/0137599 A1
(43) Pub. Date: Jun. 3, 2010

(54) PROCESS FOR PREPARING 1,4-BUTANDIOL MONITRATE

(75) Inventors: Achim Hack, Stein/ag (CH); Gunter Weingarner, Dottikon (CH); Matthias Kramer, Mellingen (CH)

Correspondence Address: ARENT FOX LLP, 1050 CONNECTICUT AVENUE, N.W., SUITE 400, WASHINGTON, DC, 20036, US

(73) Assignee: NICOX S.A., Sophia Antipolis - Valbonne (FR)

(21) Appl. No.: 12/665,664

(22) PCT Filed: Jun. 18, 2008
(86) PCT No.: PCT/EP2008/057693

§ 371 Date: Jan. 14, 2010

Related U.S. Application Data
(60) Provisional application No. 60/929,448, filed on Jun. 28, 2007.

(51) Int. Cl. C07D 491/04; C07C 205/00; C07C 69/003; C07C 69/66; C07D 491/052; C07D 231/10; C07D 207/30; C07D 261/06; C07C 229/00; C07D 487/02; C07C 381/00; C07D 209/04; C07D 333/22; C07D 209/44; C07D 209/82; C07D 313/10; C07D 337/12

(52) U.S. Cl. 546/89; 568/704; 560/129; 560/56; 560/88; 548/432; 548/375.1; 548/539; 548/247; 560/43; 548/453; 560/17; 548/491; 549/72; 548/472; 548/440; 549/354; 549/12

(57) ABSTRACT

The present invention relates to a process for the preparation of 1,4-butanediol mononitrate as intermediate for large scale preparation of high purity nitrooxybutyl ester of pharmaceutically active compounds.

### FIELD OF THE INVENTION

The present invention relates to a process for the preparation of 4-nitrooxybutan-1-ol useful as intermediate for large scale preparation of NO-releasing compounds. The present invention also concerns to a process for the preparation of NO-releasing NSAIDs using said intermediate.

### BACKGROUND TO THE INVENTION

4-nitrooxybutan-1-ol is the key intermediate in the process for the preparation of nitric oxide NO-releasing compounds, which are compounds having a —ONO₂ group linked to the pharmaceutically active molecule by a butyl linker, in said process 4-nitrooxybutan-1-ol is reacted with compounds having a carboxylic functional group, for example NSAIDs, activated either in situ or by transformation to their corresponding acid chlorides, to form nitrooxybutyl esters.

Some examples of NO-releasing compounds are (S)-2-(6-Methoxy-2-naphtyl) propionic acid 4-nitrooxybutyl ester and 2-[(2,6-dichloro phenyl)amino]benzeneacetic acid 4-(nitrooxy)butyl ester.

4-nitrooxybutyl ester of NSAIDs are in general oils or thermosoftening solids and the purification of large scale quantities of these compounds is difficult and very expensive because they cannot be crystallized, therefore for a large scale quantity production of these compounds the purity of the intermediates is one of the most important requirement for an acceptable pharmaceutical purity of the final products. In particular the 1,4-butandiol dinitrate is a critical impurity of the process for the preparation of the 4-nitrooxybutyl esters because it is carried through the entire sequence of reaction steps without any chance to reduce it.

The mononitration reactions of the 1,4-butandiol described in literature afford a mixture of 4-nitrooxybutan-1-ol and 1,4-butandiol dinitrate, the purification of the 4-nitrooxybutan-1-ol mononitrate in presence of large amount of 1,4-butandiol dinitrate is potentially dangerous in that the 1,4-butandiol dinitrate is a potentially explosive compound.

Different processes for the preparation of alkyl nitrates and 4-nitrooxybutan-1-ol have been described in the prior art. ES 2,073,995, discloses the syntheses of 3-nitratomethyl-3′-methyl-oxyethane and 3,3′-bis(nitratomethy)-oxethane from alkylsulfonates or 4-toluenesulfonates of the 3-hydroxymethyl-3′-methyl-oxyethane and of the 3,3′-bis(hydroxymethy)-oxethane and metal nitrates.

WO 04/043898 describes a process for industrial scale is production of alkanediols mononitrate using “stabilised” nitric acid. The mononitration of 1,4-butanediol according to the above cited process affords 4-nitrooxybutan-1-ol with molar yield ranging from about 30% to about 40% with a selectivity expressed as percentage ratio of 1,4-butanediol mononitrate/(1,4-butanediol mononitrate plus 1,4-butanediol dinitrate) equal to about 70-75%.

The above cited documents does not describe any method for the purification of the 1,4-butanediol mononitrate.

EP 038 862 describes a process for the preparation of diol mononitrate, said method comprises the dinitration of the diol and the subsequent transformation of the dinitrate into mononitrate using a reducing system based on hydrogen and platinum catalyst. The drawbacks of the above cited process is that the diols dinitrate are potentially high explosive and they must be handled and disposed safely.

WO 98/25918 describes the preparation of alkanediol mononitrate, said method comprises the nitration of the alkanediol followed by purification of the mononitrate derivative by chromatography or alternatively the crude nitration mixture is used without purification for the preparation of the final product. This method is less suitable for an economically feasible large scale manufacture of diol mononitrates because an expensive chromatographic purification protocol is necessary to achieve a useful quality of the subsequent products made from it. Moreover low molecular weight alkanediols mono and dinitrate are often chemical instable and they are potentially explosive, thus they must be handled with care.

The advantage of the NO-releasing compounds compared to the parent compounds are among others a good tolerance and the reduction of gastrointestinal side effects. This is especially true for NO-releasing derivatives of NSAIDs such as naproxen, diclofenac and ketoprofen.

Different processes for the preparation of 4-nitrooxybutyl ester of NSAIDs have been described in the prior art.

WO94/12463 discloses a process for the preparation of NO donating diclofenac. In said process a alkyldihalide derivates is reacted with a salt of the carboxylic acid in DMF. The reaction product is converted into the final product by reaction with AgNO₃ in acetonitrile, in accordance with literature reports. The drawbacks of the cited process is that, for a large scale production, AgNO₃ is expensive and the purification of the final product in order to obtain a pharmaceutical quality of the final product is difficult.

WO 95/09831 describes a process whereby the sodium salt of (S)-naproxen is reacted with a halo-butanol such as 4-bromobutan-1-ol or 4-chlorobutan-1-ol. The naproxen 4-hydroxybutyl ester is then halogenated in the presence of PBr₃ and the like. Alternatively, the naproxen ester is formed by reacting the sodium salt derivative with a 1,4-dihalobutane. The ester with the terminal halogen is then reacted with a nitrate source such as silver nitrate. The use of an stechiometric amount or an excess of silver nitrate to achieve a good yield of the product constitutes an economical drawback for large scale manufacturing of (S)-naproxen 4-nitrooxybutyl ester.

WO 01/10814 discloses a process for the preparation of (S)-naproxen 4-nitrooxybutyl ester with an optical purity of 97%. In said process an acid halide of (S)-naproxen is reacted with a 1,4-butandiol mononitrate in an inert organic solvent in the presence of an inorganic base, to give a (S)-naproxen 4-nitrooxybutyl ester. The 1,4-butandiol mononitrate was prepared according to the method described in WO 01/10814 cited above.

There is a need for a more selective and safer process for the preparation of large scale quantities of high pure 4-nitrooxybutan-1-ol.

### DETAILED DESCRIPTION OF THE INVENTION

The present invention provides an alternative process for the industrial preparation of 4-nitrooxybutan-1-ol in high yield and high purity under safe manufacturing conditions.

It has been found that it is possible to prepare the 4-nitrooxybutan-1-ol in good yield and substantially free of the potentially explosive 1,4-butandiol dinitrate by nitration of a mono-(C₄-C₆)alkyl ester of 1,4-butanediol containing a reduced amount of 1,4-butanediol, followed by the selective cleavage of the acid moiety of the 4-nitrooxybutan-1-ol mono-(C₄-C₆)alkyl ester.

Object of the present invention is a process for the preparation of 4-nitrooxybutan-1-ol comprising:

step a) reacting an excess of 1,4-butanediol with an acid of formula R—C(O)OH wherein R is a linear or branched (C₃-C₅)-alkyl chain, preferably R is a linear C₃ alkyl chain, in the presence of a acidic catalyst and in an aliphatic unpolar solvent, followed by selective separation of 1,4-butandiol monoester of formula (I)

RC(O)O—(CH₂)₄—OH (I)

wherein R is as above defined, from reaction mixture;
step b) nitration of 1,4-butandiol monoester with a mixture of conc. H₂SO₄ and conc. HNO₃, or a mixture of nitric acid and acetic acid or acetic anhydride, followed by isolation of the 4-nitrooxybutan-1-ol monoester of formula (II),

RC(O)O—(CH₂)₄—ONO₂ (II)

wherein R is as above defined;
step c) selective hydrolysis of the alkyl acid moiety of the compound (II) by an inorganic base, in a one layer system, followed by purification of the 4-nitrooxybutan-1-ol of formula (III)

HO—(CH₂)₄—ONO₂ (III);

said process is characterized in that the nitration step is performed using the 1,4-butandiol monoester of formula (I) containing an amount of 1,4-butandiol below 1%;

In step a) the esterification is carried out with 3 to 5 equivalent of 1,4-butanediol, the acidic catalyst is selected from the group comprising p-toluenesulfonic acid, or acid ion exchange resins, such as Dowex 50 WX2 or Amberlyst 15, in combination with p-toluenesulfonic acid. The amount of the acid catalyst ranges from about 0.003 eq. to about 0.01 eq., preferably 0.003 eq. The acidic catalyst can be recovered at the end of the reaction by filtration, in case of acidic ion exchange resins or as aqueous solution that is recycled.

In step a) the aliphatic unpolar solvent is selected from the group of aliphatic hydrocarbons comprising petrol ether fraction (80/110° C.), heptane, n-hexane, n-octane, nonane, cyclohexane, cycloheptane or a mixture thereof, preferably petrol ether (80/110° C.), n-octane or nonane.

The esterification is carried out at the reflux temperature of the solvent.

During the reaction the formed water is removed by azeotropic distillation.

In step a) the unpolar solvent plays an important role in the removal of water by azeotropic distillation and surprinsily on the selectivity of the esterification, (i.e. the ratio monoester of formula (I)/vs the diester of formula RC(O)O—(CH₂)₄—OC(O)R wherein R is as above defined) and for the removal of the diester.

The conversion of the esterification reaction of step a) is higher than 99% and the ratio of monoester (I)/diester RC(O)O—(CH₂)₄—OC(O)R) in the crude mixture is higher than 95%.

At the end of the reaction, the selective separation of 1,4-butandiol monoester from the reaction mixture of step a) comprises the following steps:
i) optionally neutralization of the crude solution of step a) by addition of a solution of sodium bicarbonate;
ii) separation of the organic phase from the unpolar solvent phase containing the 1,4-butanediol diester;
iii) extraction of the organic phase with an unpolar solvent;
iv) addition of water and a water-immiscibile chlorinated organic solvent, to the organic phase obtained in step iii) and separation of the water-immiscibile chlorinated organic solvent layer;
v) extraction of the organic phase, which contains the 1,4-butanediol monoester, with a water-immiscibile chlorinated organic solvent;
vi) washing the combined water-immiscibile chlorinated organic solvent layers of steps iv) and v) with water to remove the unreacted 1,4-butandiol to an amount below 1%, preferably below 0.5%.

The selective separation of the 1,4-butandiol monoester consists of one or more extractive cycles according to step iii) and steps v) and vi).

The unpolar solvent which can be used in step iii) is selected from the group comprising petrol ether fraction (80/110° C.), heptane, n-hexane, n-octane, nonane, cyclohexane, cycloheptane or a mixture thereof, preferably petrol ether (80/110° C.) or n-octane.

In step iv) at least 25 ml of water per mole of 1,4-butandiol is added. Typical the water-immiscibile chlorinated organic solvent of steps iv) and v) is selected from the group comprising dichloromethane, trichloromethane, tetrachloromethane, trichloroethane and tetrachloroethane, preferably dichloromethane.

In the step vi) the purified 1,4-butandiol monoester is obtained as a solution in the water-immiscibile chlorinated organic solvent and optionally is isolated by removal of the volatile solvent by vacuum evaporation.

The isolated 1,4-butandiol monoester contains a percentage of 1,4-butandiol below 1%.

The purification may be done batch wise or continuously.

In step b) the nitration reaction may be carried out batch wise in standard reaction vessels or continuously in tube reactors.

The nitration is preferably carried out by contacting the nitration mixture in a chlorinated solvent cooled to −10° C. with a solution of 1,4-butandiol monoester (I) in the same chlorinated solvent or even neat in continuous processing. The chlorinated solvent is selected from the group comprising dichloromethane, trichloromethane, tetrachloromethane, trichloroethane and tetrachloroethane, preferably dichloromethane.

The ratio of equivalents of conc. H₂SO₄ and equivalents of conc. HNO₃ ranges from 8.2:1.1 to 3:1, preferably from 5.6:1 to 3:1. Preferably in batch nitration the ratio of equivalents of conc. H₂SO₄ and equivalents of conc. HNO₃ is 8.2:1.1. Preferably in continuous nitration process the ratio of equivalents of conc. H₂SO₄ and equivalents of conc. HNO₃ ranges from 5.6:1 to 3:1.

The of ratio equivalents of conc. HNO₃ and equivalents of 1,4-butandiol monoester ranges from 1:1 to 1.6:1.

The nitration is carried out at a reaction temperature lower than +5° C., preferably between −10° C. to a maximum of +5° C.

At the end of the nitration the purification of the 4-nitrooxybutan-1-ol monoester comprises the following steps:
vii) quenching the reaction mixture with cold water or a cold water and ice and an organic chlorinated solvent and is mixing the mixture;
viii) separation of the organic chlorinated solvent phase;
ix) extraction of the quenched mixture with the organic chlorinated solvent;
x) washing the combined organic chlorinated layers with diluted sodium bicarbonate solution;
xi) washing the chlorinated organic layers with water.

The purification process of the 4-nitrooxybutan-1-ol monoester consists of one or more cycles according to steps ix) to xi).

The acid concentration after quenching is between 15% and about 60%.

The chlorinated solvent is selected from the group comprising dichloromethane, trichloromethane, tetrachloromethane, trichloroethane and tetrachloroethane, preferably dichloromethane.

The 4-nitrooxybutan-1-ol monoester may be isolated by removal of the chlorinated solvent by vacuum evaporation.

The isolated 4-nitrooxybutan-1-ol monoester is obtained in chemical yield from 80 to 90% and it contains a percentage of 1,4-butandiol dinitrate below 1%, preferably about 0.5%, and have a purity of from 95 to 97%.

In step c) the selective hydrolysis of the alkyl acid moiety (RCOOH)) of the compound of formula (II) is carried out in a one layer system consisting of a solution of the compound (II) in a low molecular weight aliphatic alcohol and water.

The hydrolysis reaction comprises the portionwise addition of the aqueous solution of the base to a solution of the 4-nitrooxybutan-1-ol monoester.

The low molecular weight aliphatic alcohol is selected from the group consisting of methanol, ethanol, the propanols, the butanols or a mixture thereof, preferably the aliphatic alcohol is methanol or ethanol.

The inorganic base is selected from the group comprising aqueous solution of sodium hydroxide, of potassium hydroxide or of lithium hydroxide; preferably a solution of sodium hydroxide or lithium hydroxide. The concentration of the base in the aqueous solution ranges between 10% to 30% of sodium hydroxide or saturated lithium hydroxide. A slight excess of base is needed to complete the reaction, the ratio of eq. of base and eq. of 4-nitrooxybutan-1-ol monoester ranges from 1.1:1 to 1.4:1. The temperature may vary in the range of from 0 to 40° C.

At the end of the reaction, the pH of the reaction mixture is adjusted to about neutral condition, such as pH 6 to 8, with an inorganic acid selected from sulphuric acid, phosphoric acid, chloridric acid, preferably sulphuric acid.

The alcoholic solvent is then distilled at a temperature that is compatibile with the thermally safe manufacture and avoiding long time heating.

The residual aqueous solution is diluted with water to about a 6% solution in water; the impurities as oily phase is then removed by phase separation, optionally using a centrifuge and/or optionally by filtration over charcoal.

Optionally the impurity as oily phase can be removed by one or more extractive cycles using an unpolar solvent which is selected from the group comprising petrol ether fraction (80/110° C.), pentane, n-hexane, heptane, n-octane, preferably n-hexane.

The residual solution containing 4-nitrooxybutan-1-ol is then subjected to a subsequent purification process comprising the following steps:
xii) extraction of 4-nitrooxybutan-1-ol into a chlorinated organic solvent immiscible with water;
xiii) washing the combined chlorinated organic layers with an aqueous solution of sodium bicarbonate;
xiv) washing the combined chlorinated organic layers with water and drying over sodium sulphate.

The purification process of the 4-nitrooxybutan-1-ol consists of one or more extractive cycles according to step xii) to xiv).

The chlorinated organic solvent immiscible with water of step xii) is selected from the group comprising dichloromethane, trichloromethane, tetrachloromethane, trichloroethane and tetrachloroethane, preferably is dichloromethane.

Optionally the purified 4-nitrooxybutan-1-ol organic solution can be concentrated to a concentration of about 15% w/w.

The resulting organic solution contains highly pure 4-nitrooxybutan-1-ol, typically above 97% pure, and methanol content about 0.05% to 0.10%.

A preferred embodiment of the present invention relates to a process for the preparation of 4-nitrooxybutan-1-ol comprising:

step a) reacting 3 to 5 eq. of 1,4-butanediol with butyric acid in petrol ether fraction (80-110° C.) or n-octane in the presence of catalytic amount of p-toluensolfonic acid and selective separation of 1,4-butandiol monobutyrate of formula (I′)

CH₃(CH₂)₂C(O)O—(CH₂)₄—OH (I′)

step b) nitration of CH₃(CH₂)₂O(O)O—(CH₂)₄—OH with a mixture of conc. H₂SO₄ and conc. HNO₃, followed by isolation of the 4-nitrooxybutyl-1-ol butyrate of formula (II′),

CH₃(CH₂)₂O(O)O—(CH₂)₄—ONO₂ (II′)

step c) selective hydrolysis of the butyric acid moiety of the 4-nitrooxybutyl-1-ol butyrate by an aqueous solution of sodium hydroxide 30%, followed by purification of the reaction mixture to obtain the 4-nitrooxybutan-1-ol of formula (III)

HO—(CH₂)₄—ONO₂ (III)

said process is characterized in that the nitration step is performed using the 1,4-butandiol monobutyrate containing an amount of 1,4-butandiol from 0.5% to 1%, or preferably below 0.5%.

In step a) the amount of the acid catalyst ranges from 0.003 eq. to about 0.01 eq., preferably 0.003 eq.

The esterification is carried out at the reflux temperature of the solvent and the formed water is removed by azeotropic distillation.

In step a) at the end of the reaction, the separation of 1,4-butandiol monobutyrate from the reaction mixture comprises the following steps:
i) optionally neutralization of the crude solution by addition of a solution of sodium bicarbonate or only by dilution with water;
ii) separation of the organic phase from the petrol ether fraction (80-110° C.) or n-octane phase;
iii) extraction of the organic phase with petrol ether fraction (80-110° C.);
iv) addition of water and a dichloromethane to the organic phase obtained in step iii) and separation of the dichloromethane phase;
v) extraction of the organic phase with dichloromethane;
vi) washing the combined dichloromethane layers with water to remove the unreacted 1,4-butandiol to an amount from 0.5% to 1% or preferably below 0.5%.

The selective separation of the 1,4-butandiol monobutyrate consists of one or more extractive cycles according to step iii) and steps v) to vi). Preferably the selective separation consists of three extractive cycles according to steps iii) and v).

In step iv) at least 25 ml of water per mole of 1,4-butandiol is added.

In the step vi) the purified 1,4-butandiol monobutyrate is obtained as a solution in dichloromethane and may optionally be isolated by removal of the volatile solvent by vacuum evaporation.

The isolated 1,4-butandiol monobutyrate contains a percentage of 1,4-butandiol below 1% and it is obtained with a chemical yield of about 80% to 90%.

The purification may be done batch wise or continuously.

In step b) the nitration is carried out by contacting the nitration mixture in dichloromethane cooled to −10° C. with a solution of 1,4-butandiol monobutyrate (I) in dichloromethane or neat in continuous processing.

The ratio of equivalents of conc. H₂SO₄ and equivalents of conc. HNO₃ ranges from 8.2:1.1 to 3:1, preferably from 5.6:1 to 3:1.

Preferably in batch nitration the ratio of equivalents of conc. H₂SO₄ and equivalents of conc. HNO₃ is 8.2:1.1. Preferably in nitration continuous process the ratio of equivalents of conc. H₂SO₄ and equivalents of conc. HNO₃ ranges from 5.6:1 to 3:1.

The ratio of equivalents of conc. HNO₃ and equivalents of 1,4-butandiol monoester ranges from 1:1 to 1.6:1.

The nitration is carried out at a reaction temperature lower than +5° C., preferably between −10° C. to a maximum of +5° C.

At the end of the nitration the purification of the 4-nitrooxybutan-1-ol butyrate comprises the following steps:
vii) quenching the crude solution with cold water and dichloromethane or cold water/ice and dichloromethane and mixing the mixture;
viii) separation of the dichloromethane phase which contains 4-nitrooxybutan-1-ol butyrate;
ix) extraction of the quenched mixture with dichloromethane;
x) washing the combined dichloromethane phases with diluted sodium bicarbonate solution;
xi) washing the combined dichloromethane phases solution with water.

The purification process of the 1,4-butandiol butyrate consists of one or more cycles according to steps ix) to xi).

The acid concentration after quenching is between 15% and about 60%.

The 4-nitrooxybutan-1-ol butyrate may be isolated by removal of the dichloromethane by vacuum evaporation.

The isolated 4-nitrooxybutan-1-ol butyrate is obtained in chemical yield of 80 to 90% and it has a chemical purity of 95-97%.

In step c) the selective hydrolysis of the butyric acid moiety of the 4-nitrooxybutan-1-ol butyrate is carried out in a one layer system consisting of a mixture of methanol and water in a ratio of about 4:1 (MeOH/water).

The selective hydrolysis consists of the portionwise addition of the aqueous solution of the base to a solution of the 4-nitrooxybutan-1-ol butyrate in methanol/water.

The inorganic base is an aqueous solution of sodium hydroxide or of potassium hydroxide or of lithium hydroxide; preferably a solution of sodium hydroxide 10% to 30%. The ratio of equivalents of base and eq. of 4-nitrooxybutan-1-ol butyrate is 1.1:1. The temperature may varied in the range of from 0 to 40° C.

At the end of the reaction, 4-nitrooxybutan-1-ol is purificated according to the process comprising the following steps:
xii) the pH of the reaction mixture is adjusted to about neutral condition, such as pH 6 to 8, with sulphuric acid;
xiii) methanol is distilled at a temperature that is compatible with the thermally safe manufacture and avoiding long time heating;
xiv) the residual aqueous solution is diluted with water to about a 6% solution in water, the oily phase containing the impurities is then removed by phase separation, optionally using a centrifuge, optionally by filtration over charcoal or optionally by one or more extractive cycles using an unpolar solvent selected from petrol ether fraction (80/110° C.), pentane, n-hexane, heptane or n-octane, preferably n-hexane;
xv) extraction of aqueous solution obtained in step xiv) with dichloromethane;
xvi) washing the combined dichloromethane phases with an aqueous solution of sodium bicarbonate;
xvii) washing of the combined dichloromethane phases with water and drying over sodium sulphate.

The purification process of the 4-nitrooxybutan-1-ol consists of one or more extractive cycles according to steps xv) to xvii).

Optionally the purified 4-nitrooxybutan-1-ol organic solution can be concentrated to a concentration of about 15% w/w.

The resulting organic solution contains 4-nitrooxybutan-1-ol, typically having a chemical purity of 97%, and methanol content below 0.05%.

The process of the invention allows to obtain the 1,4-butandiol (C₄-C₆)-alkyl monoesters, as intermediates for the nitration reaction, in advantageous yields from the industrial standpoint, therefore the nitration step of the 1,4-butandiol-(C₄-C₆)-alkyl monoesters is definitely less hazardous than the directed mononitration of 1,4-butandiol known in the art that affords a mixture of 1,4-butandiol mononitrate and the explosive 1,4-butandiol dinitrate.

It has been found that, unexpectably, the selective separation of 1,4-butandiol monoester from the unreacted 1,4-butandiol and the 1,4-butandiol diester by extraction is made possible only when the 1,4-butandiol is reacted with a (C₄-C₆)-alkyl acid.

The esterification of 1,4-butandiol with acetic or propionic acid leads to mixtures of components which cannot be separated by extractions with different solvents, for example the 1,4-butandiol monoacetate and 1,4-butandiol are too similar to achieve a good separation and to get pure 1,4-butandiol monoacetate by extraction with different solvents.

The esterification of 1,4-butandiol with long-chain alkyl acids allows a good separation of the water soluble diol, but the separation of the monoester from the diester is not feasible.

The best results were obtained by the mono-protection of the 1,4-butandiol using butyric acid for the esterification.

The selective hydrolysis of the (C₄-C₆)-alkyl acid of the 4-nitrooxybutan-1-ol (C₄-C₆)-alkyl monoester occurs under standard conditions and it affords the 4-nitrooxybutan-1-ol in high purity (above 99%). The high purity of the 4-nitrooxybutan-1-ol is a very important factor for the industrial scale preparation of the 1,4-butandiol mononitrate derivatives of active principle having a pharmaceutical acceptable purity; for example the 4-nitrooxybutyl ester of naproxen is an oil and therefore its purity depends on the obtained purity of the starting materials like the 1,4-butandiol mononitrate.

Another object of the present invention relates to a process for the preparation of nitrooxybutyl ester of NSADs of formula (IV),

M-C(O)O—(CH₂)₄—ONO₂ (IV)

wherein M is as reported below, said process comprises the reaction of a acid chloride derivative of formula (V) with 4-nitrooxybutan-1-ol of formula (III)

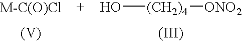

wherein M is selected from the group comprising:

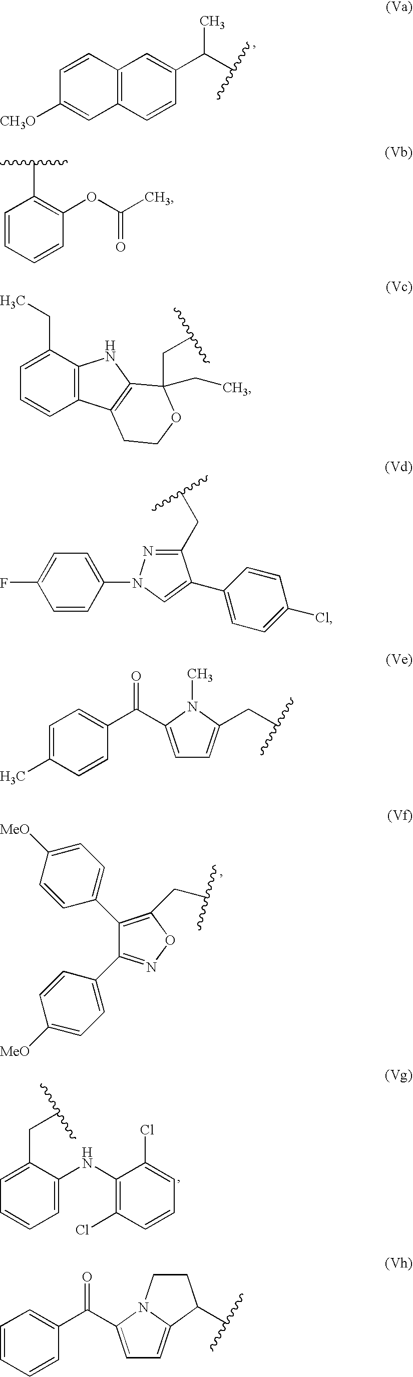

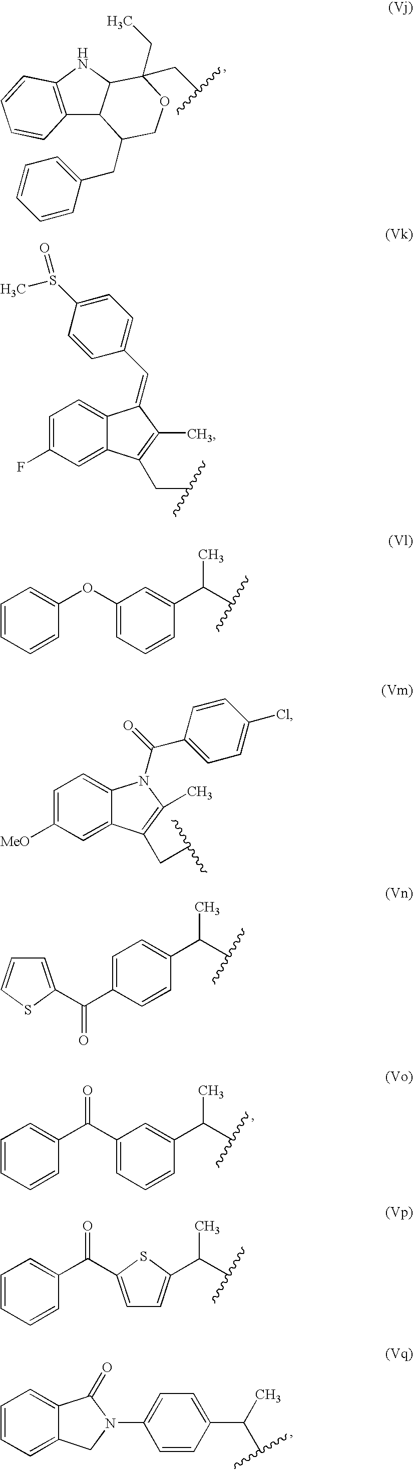

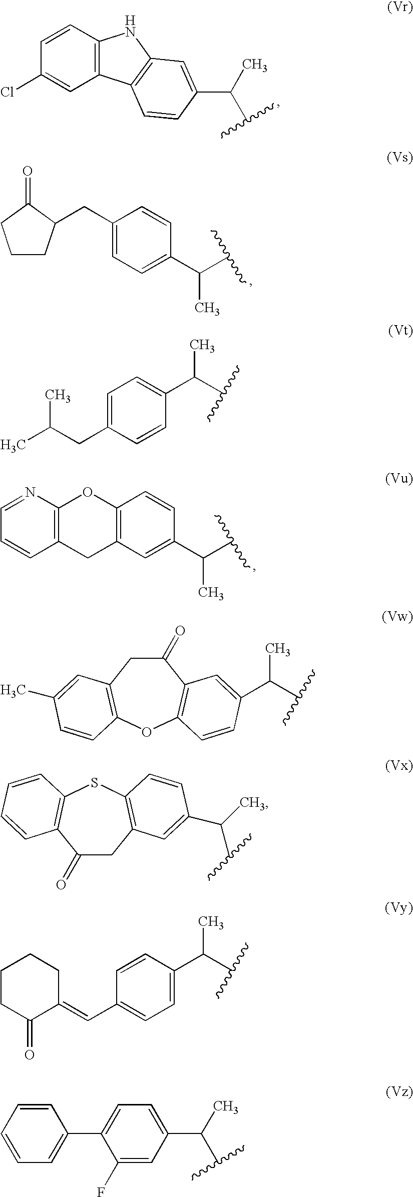

said process is characterized in that the compound of formula (III) is obtained according to the process above described.

The acid chloride of formula (V) is prepared from its corresponding acid using method described in WO 01/10814 or according to methods known in the art.

The esterification is preferably carried out by the portionwise addition of a solution of 4-nitrooxybutan-1-ol in a water immiscible solvent to a solution of the acid chloride (VI) in is the same solvent.

The esterification is carried out at a range temperature of about −2 to 40° C., preferably the coupling is performed at 40° C. or at room temperature.

A slightly excess of 4-nitrooxybutan-1-ol is used, preferably 1.1 eq. of 4-nitrooxybutan-1-ol.

The water immiscible solvent is selected from the group comprising dichloromethane, trichloromethane, tetrachloromethane, trichloroethane and tetrachloroethane, preferably dichloromethane.

At the end of the reaction, the crude mixture is first treated with water to extract the formed HCl; the organic phase is separated and then concentrated. The resulting solution is subjected to the subsequent purification which comprises the following steps:
xv) optionally washing with a solution of potassium carbonate;
xvi) extraction with water and potassium hydroxide;
xvii) washing with water;
xviii) removal of the solvent to a suitable volume;
xix) washing with a water solution of sodium chloride (1%);
xx) adding a water immiscible solvent and filtration in the presence of a filter aid optionally drying over drying agent.

The purification process consists of one or more cycles according to steps xiv) to xviii).

The purified 4-nitrooxybutyl-NSAID (IV) can be isolated by removing the solvent by vacuum evaporation.

Optionally if the compound of formula (IV) is an oil at ambient temperature, the oily compound is dissolved and the resulting solution is filtered to remove unspecific solids and the volatiles including residual water are removed by distillation.

Alternatively, if compound of formula (IV) is a solid at ambient temperature, it may be purified by crystallisation after extractive work up. The crystallisation is performed using a suitable organic solvent, if necessary an antisolvent may be used.

Suitable drying agents are anhydrous inorganic salts such as for example sodium sulphate.

Another embodiment of the invention relates to a process for the preparation of 2-(S)-(6-methoxy-2-naphtyl)-propanoic acid 4-nitrooxybutyl ester of formula (VII)

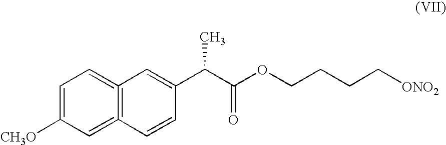

by reacting 2-(S)-(6-methoxy-2-naphtyl)-propanoyl chloride of formula (Va′)

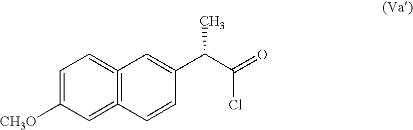

with 4-nitrooxybutyl-1-ol, said process is characterized in that the 4-nitrooxybutyl-1-ol is obtained according to the process described above;

The synthesis of the compound (Va′) may be performed in toluene with 2 to 1.2 equivalents of thionyl chloride and a catalytic amount of triethylamine. Preferably the amount of thionyl chloride is 1.2 eq.

The ratios of thionyl chloride and triethylamine ranges from 1:0.002 to 1:0.005 (eq./eq.), preferably is 1.2:0.005 (eq./eq.), more preferably is 1.05:0.005 (eq./eq.).

The reaction is carried out at a reaction temperature from 60° C. to 65° C.

The 2-(S)-(6-methoxy-2-naphtyl)-propanoyl chloride is isolated by cristallisation from the mother liquor.

In the esterification the 2-(S)-(6-methoxy-2-naphtyl)-propanoyl chloride is dissolved in dichloromethane and the solution is heated to 40° C., then a solution of 4-nitrooxybutan-1-ol solution in dichloromethane is added portion wise over a 1 hour. The resulting solution is stirred under reflux until the conversion is about 90%.

The excess of 4-nitrooxybutan-1-ol ranges from 1.05 to 1.1 eq.

Before starting the purification process of the (S)-2-(6-methoxy-2-naphtyl)propionic acid 4-nitrooxybutyl ester, the crude mixture is first treated with water to extract the formed HCl; the dichloromethane solution is separated and then concentrated. The resulting solution is subjected to the subsequent purification which comprises:

step xviii) extraction with water and potassium hydroxide;
step xix) removal of the solvent to a suitable volume of the solution;
step xx) extraction with a solution of sodium chloride (1%);
step xxi) addition of dichloromethane and filtration in the presence of a filter aid;

The purified 4-nitrooxybutyl 2-(S)-(6-methoxy-2-naphtyl)-propanoate is isolated by removing the dichloromethane by is vacuum evaporation, the oily compound is dissolved and the resulting solution is dried and the solvent is removed by distillation.

In one embodiment of the present invention the purification process of the 4-nitrooxybutyl ester consists of one or more extractive cycles according to step xviii), preferably the extractive cycles according to step xviii) are four.

A further embodiment of the invention relates to use of 4-nitrooxybutan-1-ol monoester of formula (II)

RC(O)O—(CH₂)₄—ONO₂ (II)

wherein R is a C₃-C₅ alkyl chain, as intermediate for the preparation of 4-nitrooxybutan-1-ol.

A further object of the invention is 4-nitrooxybutan-1-ol butyrate substantially free of 1,4-butanediol, i.e the content of 1,4-butanediol is below to 1%, and its use as intermediate for the preparation of 4-nitrooxybutan-1-ol.

Another advantage of the process of the present invention is that, when the active principle has one of more asymmetric atoms, the nitrooxybutyl derivatives of formula (IV) has the same optical purity (enantiomeric or diastereoisomeric purity) of the starting active principle.

### EXAMPLES

### Example 1

### Preparation of 1,4-butandiol monobutyrate (a compound of formula IIa)

Toluensulfonic acid monohydrate (2.12 g, 11.1 mmol), butyric acid (368 mL, 4.00 mol) 1,4-butanediol (1067 mL, 12.00 mol) and n-octane (750 mL) were mixed in a 2.5 L reaction vessel and the resulting emulsion was stirred vigorously at reflux for 1 h during which the water formed was removed by azeotropic distillation of the n-octane-water azeotrop. The mixture was allowed to cool to room temperature and the octane-layer was separated from the butanediol-layer containing the product and the latter phase was extracted four times with petrol ether 80/110 (300 mL for each extraction). The so obtained butanediol-layer was extracted three times with dichloromethane (500 mL for each extraction) and after phase separation the dichloromethane-layers were combined and washed four times with water (200 mL for each extraction). Removal of the dichloromethane by distillation and drying of the residue using a jacket temperature of 70° C. gave 499 g (78%) of the title compound having a chromatographic purity of 99.1% (gas chromatography) and a water content of 0.27%. This was used without further treatment in the synthesis of 4-nitrooxybutan-1-ol butyrate.

### Example 2

### Preparation of 4-Nitrooxybutan-1-ol butyrate (a compound of formula IIIa)

Sulfuric acid (96%, 285 mL, 5.13 mol) and dichloromethane (100 mL) were mixed and the mixture was cooled with stirring to −15° C. Nitric acid (98-99%, 2.9 mL, 0.069 mol) was added to the mixture with stirring. Another portion of nitric acid (98-99%, 26.0 mL, 0.618 mol) was then added in parallel with the above obtained 1,4-butandiol monobutyrate (103 mL, 0.624 mol) at such a rate that the inner temperature was maintained below −5° C. which took 90 min. An inner temperature of +5° C. must not be exceeded for stability reasons. Directly after the addition was finished the entire crude mixture was poured into a mixture of ice and water (2.25 kg) with efficient stirring keeping the inner temperature below +5° C. Stirring was switched off and the phases were allowed to separate. The dichloromethane-layer was saved and the aqueous layer was extracted with dichloromethane (400 mL). Phase separation was followed by washing of the combined dichloromethane-layers with 8% aqueous sodium bicarbonate solution (150 mL) and water (150 mL-portions) to pH 7-8. Removal of the dichloromethane by vacuum distillation at a jacket temperature below +40° C. (important for thermal safety reasons) gave 98.5 g (73% yield) of the title compound as a pale yellow oil. The purity according to GC was 95%.

¹H NMR (CDCl₃) δ 4.50 (t, J=6 Hz, 2H), 4.13 (t, J=6 Hz, 2H), 2.30 (t, J=7.4 Hz, 2H), 1.72-1.92 (m, 4H), 1.67 (sext, J=7.4 Hz, 2H), 4.50 (t, J=7.4 Hz, 3H);

¹³C NMR (CDCl₃) δ 173.9, 73.1, 63.6, 36.4, 25.3, 24.0, 18.8, 14.0;

IR 1732 (C═O), 1623, 1278 cm⁻¹.

### Example 3

### Preparation of 4-Nitrooxybutan-1-ol (compound Ia)

4-Nitrooxybutan-1-ol butyrate (1350 g, 95% w/w, 6.25 mol) was added at room temperature to a mixture of methanol (1930 ml) and water (515 ml). Sodium hydroxide (30%, 911 g, 6.83 mol) was added with stirring over 45 min and the resulting reaction mixture was allowed to stir at room temperature for 1 hour. Sulphuric acid (5%, 300 ml) was added which gave a pH of 7-8. Methanol was removed completely by vacuum distillation (80-110 mbar, inner temperature: 40-43° C., jacket temperature: 60-65° C.). The methanol amount in the reaction mixture was checked by GC (result: 0.04% related to 4-Nitrooxybutan-1-ol). Water (10300 ml) was added to the aqueous residue and the resulting reaction mixture was extracted 3 times with hexane (1800 ml, 650 ml, 650 ml) to remove 4-nitrooxybutan-1-ol butyrate and 1,4-dinitrooxybutane. The washed water layer was extracted 3 times with dichloromethane (3×5150 ml). The combined organic layers were washed once with saturated sodium bicarbonate solution (1280 ml) and two times with water (1280 ml, 640 ml). The resulting organic layer was concentrated to 4000 g (72.5% of theory related to 4-nitrooxybutan-1-ol butyrate) of a 15.3% w/w solution of the title compound. The purity according to GC was 99.7%.

### Example 4

### Preparation of 4-Nitrooxybutan-1-ol butyrate by a continuous process (compound IIIa)

The nitration of 1,4-butandiol monobutyrate was performed as a continuous process using mixed acid (H₂SO₄ and HNO₃) as nitrating agent [1.5 eq HNO₃ related to 4-hydroxybutyl butyrate]. The flow of mixed acid was approx. 10-12 l/h and of 1,4-butandiol monobutyrate approx. 2.4 kg/h. The two reaction streams were mixed in a static mixer, cooled in a heat exchanger (residence time: ca. 4 min.) and then quenched with water (approx. 22 kg per kg 1,4-butandiol monobutyrate). The quenched reaction mixture was extracted two times with dichloromethane (2.5 kg per kg 1,4-butandiol monobutyrate). The combined organic layers were washed once with sodium bicarbonate solution (1.5 L per kg 1,4-butandiol monobutyrate) and once with water (1.5 L per kg 1,4-butandiol monobutyrate). After removal of the dichloromethane in vacuum the title compound was isolated in 86% yield as yellow oil. The purity according to GC was 95-97% with a 1,4-dinitrooxybutane level of approx. 0.45%.

### Example 5

Preparation of 4-Nitrooxybutan-1-ol by a continuous process 1,4-butandiol monobutyrate (1350 g, 95% w/w, 6.25 mol) was added at room temperature to a mixture of methanol (1930 ml) and water (515 ml). Sodium hydroxide (30%, 911 g, 6.83 mol) was added with stirring over 45 min and the resulting reaction mixture was allowed to stir at room temperature for 1 hour. Sulphuric acid (5%, 300 ml) was added which gave a pH of 7-8. Methanol was removed completely by vacuum distillation (80-110 mbar, inner temperature: 40-43° C., jacket temperature: 60-65° C.). The methanol amount in the reaction mixture was checked by GC (result: 0.04% related to 4-Nitrooxybutan-1-ol). Water (10300 ml) was added to the aqueous residue and the resulting reaction mixture was extracted 3 times with hexane (1800 ml, 650 ml, 650 ml) to remove 4-nitrooxybutan-1-ol butyrate and 1,4-dinitrooxybutane. The washed water layer was extracted 3 times with dichloromethane (3×5150 ml). The combined organic layers were washed once with saturated sodium bicarbonate solution (1280 ml) and two times with water (1280 ml, 640 ml). The resulting organic layer was concentrated to 4000 g (72.5% of theory related to 4-nitrooxybutyl butyrate) of a 15.3% w/w solution of the title compound. The purity according to GC was 99.7%.

### Comparative Examples 6 and 7

### Preparation of 4-Hydroxybutan-1-ol acetate (Ex. 6) and of 4-Hydroxybutan-1-ol propionate (Ex. 7)

HO(CH₂)₄OH+R′COOH(Xa, Xb)+cat.->R′C(O)O(CH₂)₄OH(Ia, Ib)+R′C(O)O(CH₂)₄OC(O)R′(XIa, XIb)+H₂O

### Ia, Xa and XIa: R′=CH₃—

### Ib, Xb and XIb: R′=CH₃CH₂—

When the esterification was carried out with acetic acid (R′ is CH₃—) the obtained crude mixture generally consisted of 20% of 1,4-butanediol, 60% of 1,4-butanediol-monoacetate and 20% of 1,4-butanediol-diacetate. The 1,4-butanediol-monoacetate could not be extracted from the 1,4-butanediol/water mixture because of its polar and protic properties. Because of these difficulties the crude mixture was distilled by 70 plates laboratory column under reduced pressure (30-35 mmHg). The distillates had a costant composition containing 7% of the undesired 1,4-butanediol, the 75% of 1,4-butanediol-diacetate and the 18% of 1,4-butanediol-monoacetate. The distillation residue mainly consisted of 1,4-butanediol-monoacetate which was partly decomposed at the high temperature during distillation.

The same esterification carried out with propionic acid (R′ is CH₃CH₂—) provided a crude mixture which consisted of 38% of 1,4-butanediol, 60% of 1,4-butanediol monopropionate and 2% of 1,4-butanediol-dipropionate. The distillates had the following compositions: 25% of 1,4-butanediol, 50% of 1,4-butanediol-monopropionate and 25% of 1,4-butanediol dipropionate, and only a fraction contains the 10% of 1,4-butanediol, 85% of 1,4-butanediol-monopropionate and the 10% of 1,4-butanediol dipropionate.

The percentages of the components in the crude reaction mixtures and in the collected fractions are reported in table 1.

TABLE 1

| | Components of the | |
|---|---|---|
| R′ | crude mixture | Collected fractions |
| CH₃— | 1,4-butandiol: 20% | 1,4-butandiol: 7% |
| | Ia: 60% | Ia: 18% |
| | XIa: 20% | XIa: 75% |
| CH₃CH₂— | 1,4-butandiol: 38% | 1,4-butandiol: 25% |
| | Ib: 60% | Ib: 50% |
| | XIb: 2% | XIb: 25% |

### Example 6

### Preparation of 1,4-butanediol-monoacetate

500 ml petrolether (80/110), 733 g ethylacetate (8.32 mol), 500 g 1,4-butanediol (5.55 mol) and 40 g Amberlyst 15 were mixed in 2.5 L reaction vessel and the resulting emulsion was stirred vigorously at reflux for 24 h. Then the reaction mixture was filtered. Removal of the volatile solvents gave an oily residue (550 ml) consisting of 20% of 1,4-butanediol, 60% of 4-hydroxybutyl acetate and 20% of 1,4-butanediol-diacetate. Conversion 80%.

A further purification of the 1,4-butanediol-monoacetate by distillation failed. The collected distillate had a constant composition over time consisting of 75% of 1,4-butanediol-diacetate, 18% of 1,4-butanediol-monoacetate and 7% of 1,4-butanediol. The distillation residue contained 35% of 1,4-butanediol-diacetate, 50% of 1,4-butanediol-monoacetate and 15% of 1,4-butanediol. [Distillation conditions: 70 plate preparative laboratory column, 28-33 Torr, head temperature: 113-115° C., inner temperature: 144-145° C., jacket temperature: 170-190° C.]

### Example 7

### Preparation of 1,4-butanediol-monopropionate

750 ml petrolether (80/110), 890 g ethylpropionate (8.71 mol), 500 g 1,4-butanediol (5.55 mol) and 20 g Dowex 50 WX2 were mixed in 2.5 L reaction vessel and the resulting emulsion was stirred vigorously at reflux for 24 h. Then the reaction mixture was filtered. Removal of the volatile solvents gave an oily residue (731 g) consisting of 38% 1,4-butanediol, 60% 1,4-butanediol monopropionate and 2% 1,4-butanediol dipropionate. Conversion 62%.

The crude oil was further purified by vacuum distillation [70 plate preparative laboratory column, 28-30 Torr, head temperature: 119-121° C., inner temperature: 146-156° C., jacket temperature: 190-192° C.].

The distillation of the crude mixture yielded a 25% of 1,4-butanediol a 50% of 1,4-butanediol monopropionate and a 25% of 1,4-butanediol dipropionate and a small fraction consisting of 10% of 1,4-butanediol, 85% of 1,4-butanediol monopropionate and 10% of 1,4-butanediol dipropionate.

Only a small fraction of approx. 30 ml was obtained with a purity of >85% of the title compound.

¹H NMR (CDCl₃) δ=1.14 (t, J=7.6 Hz, 3H), 1.59-1.78 (m, 4H), 1.87 (bs, 1H), 2.33 (q, J=7.6 Hz, 2H), 3.68 (t, J=6.3 Hz, 2H), 4.11 (t, J=6.3 Hz, 2H).

### Example 8

### Preparation of (S)-2-(6-Methoxy-2-naphtyl)propionyl chloride (compound Va′)

(S)-Naproxen (compound Ia, 56 kg, 243 mol) and cyclohexane (420 L) and triethylamine (51 g, 0.50 mol) were added to a 800 L reaction vessel and the resulting suspension was stirred under nitrogen and heated to an inner temperature of 60° C. After this a parallel addition of thionyl chloride (34.7 kg, 292 mol) and a solution of triethylamine (76 g, 0.75 mol) in cyclohexane (14 L) was started and continued over 1.5 h. The solution of triethylamine was added under the liquid surface of the suspension. After the addition was finished the reaction mixture was agitated for another 30 min at an inner temperature of 60° C. after which HPLC showed full conversion. The reaction solution was filtered hot and then cooled slowly to 0° C. Crystallisation started at around 50° C. and after reaching 0° C. the slurry was stirred for another 30 min before the crystals were filtered off using a pressure filter. The crystals were washed with cyclohexane (75 L) and then dried under vacuum at 40° C. to give 54 kg (89%) of pure VIa as white crystals.

### Example 9

### Preparation of (S)-2-(6-Methoxy-2-naphtyl) propionic acid 4-nitrooxybutyl ester (compound VII)

(S)-2-(6-Methoxy-2-naphtyl)propionyl chloride (50 g, 0.20 mol) to and dichlormethane (113 mL) were added to a reaction vessel and the resulting suspension was stirred at room temperature under nitrogen for about 10 min during which the solid dissolved. The solution was heated to an inner temperature of 40° C. and 4-nitrooxybutan-1-ol (210 g of a 14.6% w/w solution of 4-nitrooxybutan-1-ol in dichlormethane, 0.231 mol) was added over about 70 min at this temperature. After the addition was finished the reaction was stirred for another 70 min before heating was stopped and water (100 mL) was added. After stirring the formed twophase system for around 6 min agitation was stopped and after another 7 min the phases were separated. The organic layer was then stirred and heated to reflux temperature and kept there for a total of 6 h. Potassium hydroxide (1 g, 0.02 mol) and water (100 mL) were added and the resulting two-phases system was agitated for about 20 min before phase separation. After repeating this extraction once more the volatiles were removed by distillation at 40° C. and around 700 mbar to give a clear yellow residue that was washed four times with water (200 mL per portion) to remove residual VIa. Sodium sulphate (1.5 g) and Harborlite (1.5 g) were added to the obtained organic liquid phase and the resulting mixture was filtered. The solids were washed with dichlormethane (40 mL) and the combined organic layer was distilled to dryness at 40° C. to give 61.19 g (88%) of pure compound (Va). Characterisation data are in accordance with the data reported in WO 01/10814.

1. A process for the preparation of 4-nitrooxybutan-1-ol comprising:
step a) reacting an excess of 1,4-butanediol with an acid of formula R—C(O)OH wherein R is a linear or branched (C₃-C₅)-alkyl chain in the presence of a acidic catalyst and in an aliphatic unpolar solvent, followed by selective separation of 1,4-butandiol monoester of formula (I)
RC(O)O—(CH₂)₄—OH (I)
wherein R is as above defined, from reaction mixture;
step b) nitration of RC(O)O—(CH₂)₄—OH wherein R is as above defined, with a mixture of cone. H₂SO₄ and conc. HNO₃, or a mixture of nitric acid and acetic acid or acetic anhydride, followed by isolation of the 4-nitrooxybutan-1-ol monoester of formula (II),
RC(O)O—(CH₂)₄—ONO₂ (II)
wherein R is as above defined;
step c) selective hydrolysis of the alkyl acid moiety of the compound (II) by an inorganic base, in a one layer system, followed by purification of the 4-nitrooxybutan-1-ol of formula (III)
HO—(CH₂)₄—ONO₂ (III);
said process is characterized in that the nitration step is performed using the 1,4-butandiol monoester of formula (I) containing an amount of 1,4-butandiol below 1%.

2. A process according to claim 1 wherein the esterification in step a) is carried out using 3 to 5 equivalent of 1,4-butanediol.

3. A process according to claim 1 wherein the acidic catalyst is selected from the group comprising p toluensulfonic acid, or acid ion exchange resins in combination with p-toluenesulfonic acid.

4. A process according to claim 1 wherein the selective separation of 1,4-butandiol monoester comprises the following steps:
ii) separation of the organic phase from the unpolar solvent phase;
iii) extraction of the organic phase with an unpolar solvent;
iv) addition of water and a water-immiscibile chlorinated organic solvent, to the organic phase obtained in step iii) and separation of the water-immiscibile chlorinated organic solvent layer;
v) extraction of the organic phase with the water-immiscibile chlorinated organic solvent;
vi) washing the combined water-immiscibile chlorinated organic solvent layers of steps v) and iv) with water to remove the unreacted 1,4-butandiol to an amount below 1%.

5. A process according to claim 4 further comprising the neutralization of the crude solution of step a) by addition of a solution of bicarbonate or by dilution with water.

6. A process according to claim 1 wherein the nitration is carried out in a chlorinated solvent.

7. A process according to claim 5 wherein the nitration is carried out at a reaction temperature between −10° C. to a maximum of +5° C.

8. A process according to claim 1 wherein the purification of the 4-nitrooxybutan-1-ol monoester in step b) comprises the following steps:
vii) quenching the crude solution with cold water and a organic chlorinate solvent or cold water/ice and a organic chlorinate solvent and mixing the mixture; vii) separation of the organic chlorinate solvent phase;
ix) extraction of the quenched mixture with chlorinate solvent;
x) washing the combined organic chlorinate solvent layers with diluted sodium bicarbonate solution;
xi) washing the combined organic chlorinate solvent layers with water.

9. A process according to claim 1 wherein the one layer solvent system in step c) is a mixture of a low molecular weight aliphatic alcohol and water.

10. A process according to claim 1 wherein the inorganic base is selected from the group consisting of aqueous solution of sodium hydroxide, or of potassium hydroxide or of lithium hydroxide.

11. A process according to claim 1 wherein in step c) the purification of 4-nitrooxybutan-1-ol comprises the followings steps:
xii) neutralizing the reaction mixture with sulphuric acid;
xiii) distillation of the low molecular weight aliphatic alcohol;
xiv) diluting the residual aqueous solution with water to about a 6% solution in water, and separation of the oily phase containing the impurities;
xv) extraction of the aqueous solution with a organic chlorinated solvent immiscible with water;
xvi) washing the combined organic chlorinated solvent layers with an aqueous solution of sodium bicarbonate;
xvii) washing the combined organic chlorinated solvent layers with water and drying over sodium sulphate.

12. A process according to claim 1 wherein R is a linear C3-alkyl chain.

13. A process according to claim 12 wherein the unpolar solvent in step a) is petrol ether fraction (80/110° C.) or n-octane.

14. A process according to claim 12 wherein the acidic catalyst is p-toluenesulfonic acid.

15. A process according to claim 14 wherein the amount of the acid catalyst ranges from about 0.003 equivalents to about 0.01 equivalents.

16. A process according to claim 13 wherein the esterification of step a) is carried out at the reflux temperature of the solvent.

17. A process according to claim 12 wherein the selective separation of 1,4-butandiol monobutyrate comprises the following steps:
ii) separation of the organic phase from the petrol ether fraction (80-110° C.) or n-octane phase;
iii) extraction of the organic phase with petrol ether fraction (80-110° C.);
iv) addition of water and dichloromethane to the organic phase obtained in step iii);
v) extraction of the organic phase with dichloromethane;
vi) washing the combined dichloromethane layers with water to remove the unreacted 1,4-butandiol to an amount below 1%.

18. A process according to claim 17 comprising more than one extraction cycles according to steps iii), v) and vi).

19. A process according to claim 12 wherein the nitration of step b) is carried out in dichloromethane with a nitration mixture of conc. H₂SO₄ and conc. HNO₃ wherein the ratio of equivalents of conc. H₂SO₄ and equivalents of conc. HNO₃ ranges from 8.2:1.1 to 3:1.

20. A process according to claim 19 wherein the ratio of equivalents of conc. H₂SO₄ and equivalents of conc. HNO₃ is from 5.6:1 to 3:1.

21. A process according to claim 19 wherein in step b) the ratio eq. conc. HNO₃ and eq. 1,4-butandiol monobutyrate ranges from 1:1 to 1.6:1.

22. A process according to claim 19 wherein the nitration is carried out at a reaction temperature between −10° C. to +5° C.

23. A process according to claim 19 wherein the purification of the 4-nitrooxybutan-1-ol butyrate in step b) comprises the following steps:
vii) quenching the crude solution with cold water and dichloromethane or cold water/ice and dichloromethane and mixing the mixture;
viii) separation of the dichloromethane phase;
ix) extraction of the quenched mixture with dichloromethane;
x) washing the combined dichloromethane phases with diluted sodium bicarbonate solution;
xi) washing the combined dichloromethane phases solution with water.

24. A process according to claim 23 comprising one or more extraction cycles according to steps ix) to xi).

25. A process according to claim 12 wherein the selective hydrolysis of the butyric acid moiety in step c) is carried out in a one layer system consisting of methanol and water in a ratio of about 4:1, and the inorganic base is a 10% to 30% aqueous solution of sodium hydroxide.

26. A process according to claim 25 wherein the ratio of eq. of the inorganic base and eq. of 4-nitrooxybutan-1-ol butyrate ranges from 1.1:1 to 1.4:1.

27. A process according to claim 25 wherein the selective hydrolysis of the butyric acid moiety of step c) is carried out at a temperature from 0 to 40° C.

28. A process according to claim 25 wherein in step c) the purification of 4-nitrooxybutan-1-ol comprises the followings steps:
xii) neutralization of the reaction mixture with sulphuric acid;
xiii) distillation of methanol;
xiv) dilution of the residual aqueous solution water to about a 6% solution in water and separation of the oily phase containing the impurities;
xv) extraction of the aqueous solution with dichloromethane;
xvi) washing the combined dichloromethane phases with an aqueous solution of sodium bicarbonate:
xvii) washing of the combined dichloromethane phases with water and drying over sodium sulphate.

29. A process according to claim 28 wherein the separation of the oily phase containing the impurities in step xiv) is carried out by one or more extractive cycles using an unpolar solvent.

30. A process according to claim 29 wherein the unpolar solvent is hexane.

31. Use of a compound of formula (II)
RC(O)O—(CH₂)₄—ONO₂ (II)
wherein R is a C₃-C₅ alkyl chain, as intermediate for the preparation of 4-nitrooxybutan-1-ol.

32. Use of a compound of formula (II) according to claim 31 wherein R is a linear C₃-alkyl chain.

33. 4-nitrooxybutan-1-ol butyrate.

34. A process for the preparation of 4-nitrooxybutan-1-ol butyrate of claim 33 comprising the following steps:
a) reacting an excess of 1,4-butanediol with a butyric acid in the presence of a acidic catalyst and in petrol ether fraction (80/110° C.) or n-octane;
ii) separation of the organic phase from the petrol ether fraction (80-110° C.) or n-octane phase;
iii) extraction of the organic phase with petrol ether fraction (80-110° C.);
iv) addition of water and dichloromethane to the organic phase obtained in step iii);
v) extraction of the organic phase with dichloromethane;
vi) washing the combined dichloromethane layers with water to remove the unreacted 1,4-butandiol to an amount below 1% and removal of the dichloromethane by vacuum evaporation;
b) nitration of the 1,4-butandiol monobutyrate obtained in step vi) with a nitration mixture of conc. H₂SO₄ and conc. HNO₃ wherein the ratio of equivalents of cone. H₂SO₄ and equivalents of conc. HNO₃ ranges from 8.2:1.1 to 3:1.
vii) quenching the crude solution with cold water and dichloromethane or cold water/ice and dichloromethane and mixing the mixture,
viii) separation of the dichloromethane;
ix) extraction of the quenched mixture with dichloromethane;
x) washing the combined dichloromethane phases with diluted sodium bicarbonate solution;
xi) washing the combined dichloromethane phases solution with water.

35. A process for the preparation of a compound of formula (IV)
M-C(O)O—(CH₂)₄—ONO₂ (IV)
wherein M is as reported below, comprising reacting an acid chloride derivative of formula (V) with 4-nitrooxybutan-1-ol of formula (III) 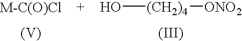
wherein M is selected from the group comprising: 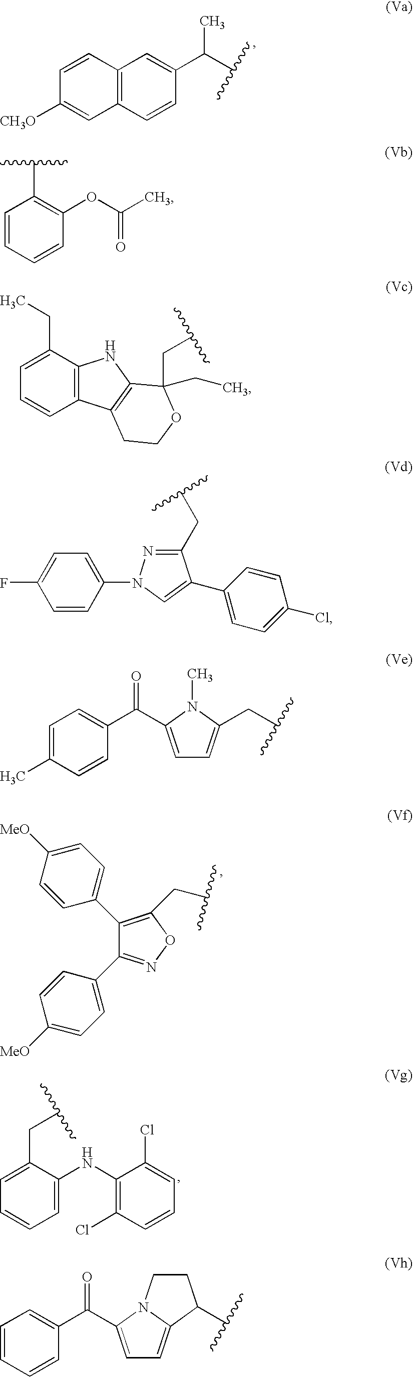 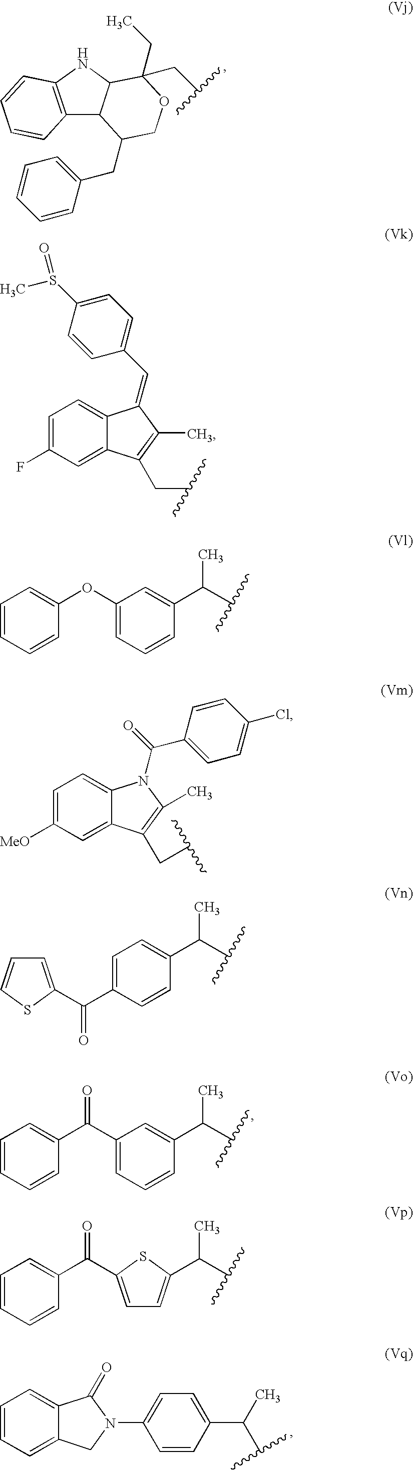 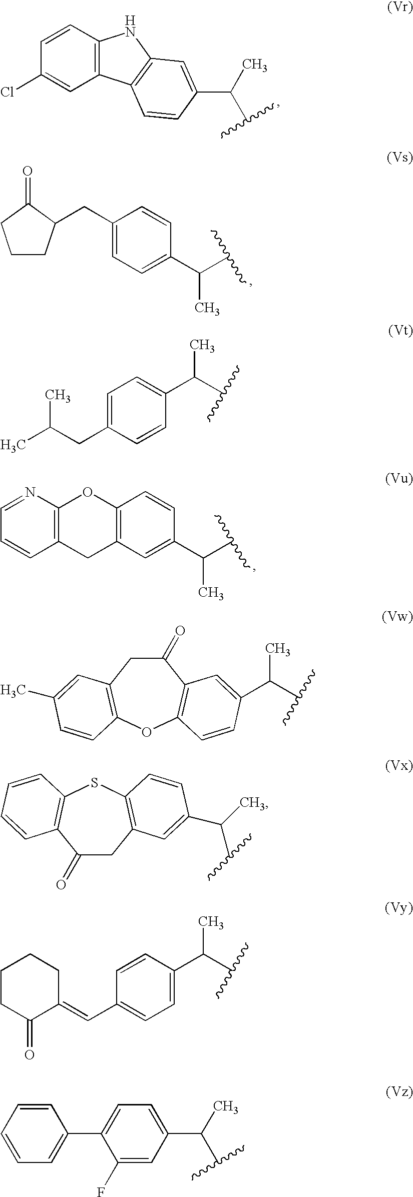
said process is characterized in that the compound of formula (III) is prepared according to claim 1.

36. A process according to claim 35 for the preparation of 2-(S)-(6-methoxy-2-naphtyl)-propanoic acid 4-nitrooxybutyl ester of formula (VII) 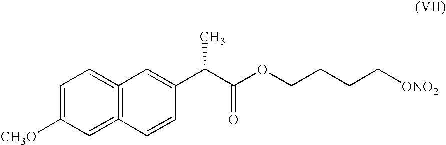
comprising reacting the 2-(S)-(6-methoxy-2-naphtyl)-propanoyl chloride of formula (Va′) 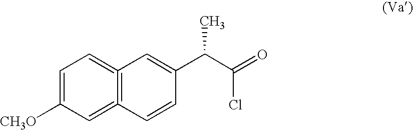
with 4-nitrooxybutan-1-ol of formula (III) prepared according the claims 12 to 28.
